(19) United States
(12) Patent Application Publication
(10) Pub. No.: US 2026/0363639 A1
(43) Pub. Date: Nov. 19, 2026

(54) SIGNAL PROCESSING DEVICE, SIGNAL PROCESSING SYSTEM, AND SIGNAL PROCESSING METHOD

(71) Applicant: Hitachi, Ltd., Tokyo (JP)

(72) Inventor: Hiroshi OINUMA, Tokyo (JP)

(21) Appl. No.: 19/536,303

(22) Filed: Feb. 11, 2026

(30) Foreign Application Priority Data
May 16, 2025 (JP) 2025-082322

(51) Int. Cl. A61B 6/42

(52) U.S. Cl. CPC A61B 6/4241

(57) ABSTRACT

Provided is a signal processing device capable of improving detection accuracy. A signal state determination unit determines whether or not an electrical signal is continuous within an added time obtained by adding a predetermined determination time to time information of a peak value, and an output unit outputs at least one of a photon-counting-type signal and an integration-type signal based on a state signal from the signal state determination unit.

### CROSS-REFERENCE TO RELATED APPLICATION

The present application claims priority from Japanese application JP2025-082322, filed on May 16, 2025, the content of which is hereby incorporated by reference into this application.

### BACKGROUND OF THE INVENTION

### 1. Field of the Invention

The present invention relates to a signal processing device, a signal processing system, and a signal processing method.

### 2. Description of the Related Art

An optical signal detector is an important device for converting an optical signal into an electrical signal, and devices, such as a silicon photomultiplier (SiPM) and a photomultiplier tube (PMT), are widely used. These devices are increasingly applied in diverse fields, such as medicine, communications, and scientific research, and play an essential role particularly in detecting weak optical signals.

For optical signal detection circuits, the following methods are generally known: an integration method that integrates an output pulse signal of an optical signal detector within a specific time range; and a photon counting method that treats each pulse as a discrete pulse and measures the number of photons from the peak value of a signal.

The photon counting method can accurately measure extremely weak light. However, when the amount of light increases and individual pulses overlap (pile up), it is not possible to accurately measure the light. In addition, when the amount of light increases further, the signal reaches a saturated state, and a pulse signal is zero. As a result, there is a possibility of missed signal detection.

On the other hand, the integration method is inferior to the photon counting method in detection accuracy at low light levels, but has the advantage of being able to stably measure light even at high light levels.

Therefore, in order to cover a wide range of light levels, a method is generally used that prepares signal processing circuits adapted to each dynamic range and switches between signal processing methods according to the expected light level.

As this signal processing technique, for example, a reading circuit disclosed in JP 2017-83298 A is known. The system is a circuit that reads an output current from a photoelectric conversion element, and includes a photon counting circuit that counts the number of photons incident on the photoelectric conversion element, an integration circuit that integrates a current to generate a voltage signal, and a signal processing unit that determines the intensity of light incident on the photoelectric conversion element, based on the counting result output from the photon counting circuit and the magnitude of the voltage signal output from the integration circuit.

### SUMMARY OF THE INVENTION

In the optical signal detection circuit, in order to cover a wide range of light levels, a method is generally used that prepares signal processing circuits adapted to each dynamic range and switches between the signal processing circuits based on a threshold value according to the light level.

However, when the detection circuits are switched in real time based on the threshold value for samples from which the detected signal quantities are unknown, there is a possibility that detection accuracy may be reduced due to false detection or missed signal detection.

An object of the present invention is to provide a signal processing device capable of improving detection accuracy.

According to an aspect of the present invention, there is provided a signal processing device for processing an electrical signal output from an optical signal detector using a processor. The signal processing device includes a peak detection unit, a photon-counting-type signal processing unit, an integration-type signal processing unit, a time extraction unit, a signal state determination unit, and an output unit. The peak detection unit detects a peak value of the electrical signal. the photon-counting-type signal processing unit detects the peak value included in the electrical signal in a predetermined section to detect the number of photons in the predetermined section and outputs a photon-counting-type signal. The integration-type signal processing unit integrates a signal included in the electrical signal in the predetermined section to measure a signal quantity and outputs an integration-type signal. The time extraction unit holds time information of the peak value detected by the peak detection unit. The signal state determination unit determines whether or not the electrical signal is continuous within an added time obtained by adding a predetermined determination time to the time information of the peak value. The output unit outputs at least one of the photon-counting-type signal and the integration-type signal, based on a state signal from the signal state determination unit.

According to an aspect of the present invention, it is possible to provide a signal processing device capable of improving detection accuracy.

### BRIEF DESCRIPTION OF THE DRAWINGS

FIG. 1 is a schematic diagram showing an example of a configuration of a signal processing device according to Example 1;

FIG. 2 is a diagram showing an example of display content of an image display unit;

FIG. 3 is a conceptual diagram showing an example of content of a process of switching between signal processing methods based on a threshold value;

FIG. 4 is a conceptual diagram showing an example of content of a process of switching between the signal processing methods using time information of a signal in the signal processing device according to Example 1;

FIG. 5 is a flowchart showing a signal processing method according to Example 1; and

FIG. 6 is a schematic diagram showing an example of a configuration of a signal processing device according to Example 2.

### DETAILED DESCRIPTION OF THE PREFERRED EMBODIMENTS

Hereinafter, embodiments of the present invention will be described.

First, an example of false detection and missed detection will be described with reference to FIG. 3. A waveform shown in FIG. 3 is a pulse waveform output by an optical signal detector.

In section 1, a waveform peak is observed, and a peak value H0 is measured as a measured value by a photon counting method. In section 2, pile-up occurs due to superimposition on a pulse signal in section 1, resulting in a pulse waveform that exceeds a threshold value.

In this case, since the pulse waveform is observed, it can be accurately detected from the pulse height by the photon counting method. However, since the pulse waveform exceeds the threshold value, it becomes an integrated value at a sampling point in section 2 and includes a signal noise component. As a result, detection accuracy is reduced.

In addition, when the pulse waveform does not exceed the threshold value, but piles up and becomes a waveform resembling a direct-current signal as shown in section 4, there is a possibility that an undetected location may occur, resulting in a reduction in detection accuracy.

An embodiment of the present invention provides a signal processing device capable of improving detection accuracy.

Hereinafter, examples will be described with reference to the drawings.

### Example 1

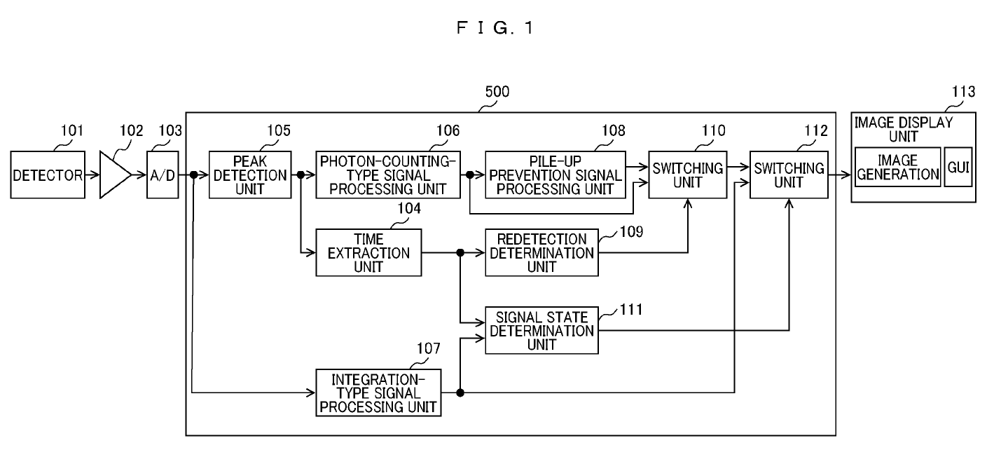

FIG. 1 is a schematic diagram showing an example of a configuration of a signal processing device and a signal processing system according to Example 1.

As shown in FIG. 1, the signal processing system according to Example 1 includes an optical signal detector 101, a preamplifier circuit 102, an A/D converter 103, a signal processing device 500, and an image display unit 113.

The optical signal detector 101 converts photons into an electrical signal and transmits the electrical signal to the preamplifier circuit 102. For example, the optical signal detector 101 may be a silicon photomultiplier (SiPM) or a photomultiplier tube (PMT).

The preamplifier circuit 102 amplifies a weak signal from the optical signal detector 101. At that time, a filtering circuit (not shown) removes noise from the signal. The A/D converter 103 converts the electrical signal from the preamplifier circuit 102 into digital data.

The signal processing device 500 processes the electrical signal from the A/D converter 103 to form a detection image. The image display unit 111 displays the detection image formed by the signal processing device 500.

The signal processing device 500 according to Example 1 includes a time extraction unit 104, a peak detection unit 105, a photon-counting-type signal processing unit 106, an integration-type signal processing unit 107, a pile-up prevention signal processing unit 108, a redetection determination unit 109, a switching unit 110, a signal state determination unit 111, and a switching unit 112.

The peak detection unit 105 detects a signal peak value of the digital data from the A/D converter 103. For example, the peak detection unit 105 detects the timing of a zero-crossing point at which a sign of derivative data changes from positive to negative, based on the derivative data of the signal. Therefore, peaks included in the digital data from the A/D converter 103 and in the electrical signal from the optical signal detector 101 are detected.

The photon-counting-type signal processing unit 106 detects a peak value included in the electrical signal from the peak detection unit 105 in a predetermined section to detect the number of photons (photon count) in the predetermined section. When there are a plurality of peak values in the predetermined section, the total number of photons in the predetermined section is calculated by simple addition or the like.

The integration-type signal processing unit 107 integrates signals included in the digital data from the A/D converter 103 and in the electrical signal from the optical signal detector 101 in a predetermined section to measure a signal quantity. The time extraction unit 104 holds time information of the peak value extracted by the peak detection unit 105.

The pile-up prevention signal processing unit 108 outputs a pile-up prevention signal obtained by subtracting a pile-up error from a photon-counting-type signal from the photon-counting-type signal processing unit 106.

The redetection determination unit 109 holds the time information of the peak value held by the time extraction unit 104 and a time obtained by adding a determination time T' to the time information of the peak value, and detects the generation of a new pulse within the time. Here, the determination time T' is a value that can be set by a user. For example, the fall time of an output pulse, which is estimated from the detector or the preamplifier circuit and may vary depending on the model or the individual device, is set as the determination time T'.

The switching unit 110 switches between the results of the photon-counting-type signal processing unit 106 or the pile-up prevention signal processing unit 108, based on the signal from the redetection determination unit 109.

The signal state determination unit 111 determines whether or not the data from the integration-type signal processing unit 107 is continuous during the period between the time information of the peak value and the determination time T’ output by the time extraction unit 104.

In this case, whether or not the data is continuous may be determined by, for example, determining whether or not the output data of the integration-type signal processor 107 is a value exceeding a determination width d from the initial data of the time information of the peak value + the determination time T' output by the time extraction unit 104 based on a parameter called the determination width d. In this case, the determination width d may be set to a value that takes into account the width of detector noise and circuit noise.

The switching unit 112 switches between the signal from the switching unit 110 and the signal from the integration-type signal processing unit 107, based on the signal from the signal state determination unit 111.

The image display unit 113 generates a detection image based on the signal output by the switching unit 112 and displays the detection image on the screen, using an image generation process and a GUI. In addition, for example, the image display unit 113 can display a setting screen (graphical user interface (GUI)) based on a process of a setting unit (not shown) provided in the signal processing device 500.

When an image is generated in the image generation process, the pixel value of a pixel is calculated, for example, the peak values of peak positions included in a section corresponding to one pixel are integrated or averaged to calculate the pixel value of the pixel.

In addition, a processor, such as a central processing unit (CPU), a graphics processing unit (GPU), or a digital signal processor (DSP), executes a program to implement the signal processing device 500 and the image display unit 113 shown in FIG. 1. Alternatively, the signal processing device 500 and the image display unit 113 may be implemented by hardware, such as a field programmable gate array (FPGA) or an application specific integrated circuit (ASIC), or may be implemented by a combination of hardware and software.

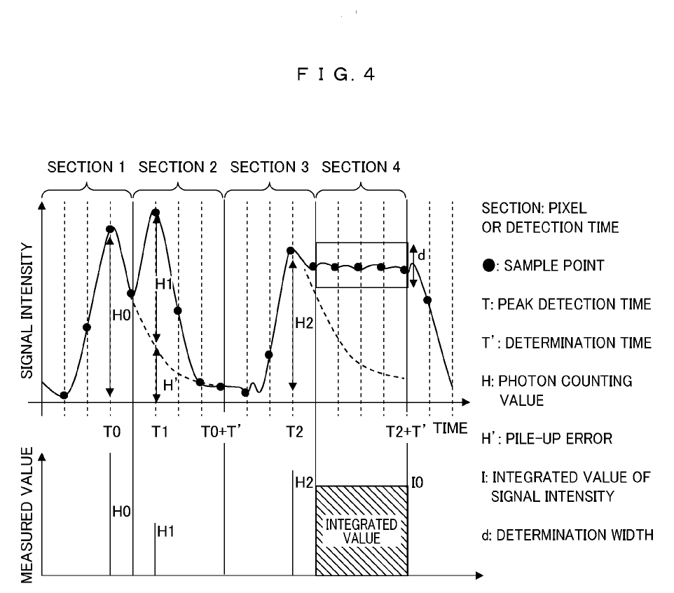

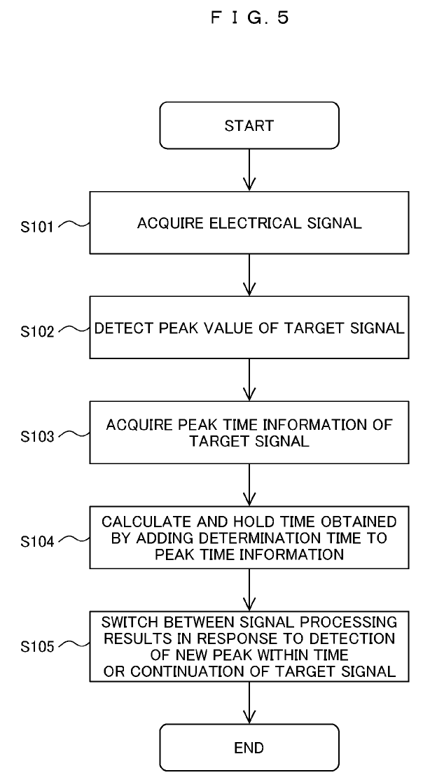

FIG. 4 is a conceptual diagram showing an example of the content of a process of switching signal processing using the time information of the signal in the signal processing device 500 according to Example 1. FIG. 4 is a diagram showing the results of the switching between photon-counting-type signal processing and integration-type signal processing.

FIG. 4 shows an example of time-series changes in the digital data from the A/D converter 103 and the electrical signal from the optical signal detector 101.

In FIG. 4, T0, T1, and T2 are peak times (peak timings) detected by the peak detection unit 105 for the electrical signal. Sections 1 to 4 are predetermined sections (pixels or detection times). Here, an example in which the section is treated as one pixel and four sample points are treated as one pixel will be described.

In section 1, a peak position T0 is detected, and H0, which is the peak value of the peak position T0, is sent to the photon-counting-type signal processing unit 106. The photon-counting-type signal processing unit 106 adds the peak values in section 1 and outputs the added value. In section 1, the switching of signal processing is not performed, and H0 is output as the pixel value to the image display unit 113.

In section 2, a peak position T1 is measured. At this time, the time extraction unit 104 extracts T0 when the peak position T0 is measured, and the redetection determination unit 109 calculates T0+T' using the determination time T'. At this time, the redetection determination unit 109 determines whether or not a peak has occurred within the section T0+T'.

The pile-up prevention signal processing unit 108 calculates the fall time of the signal from the signal output by the photon-counting-type signal processing unit 106 using a model waveform at any time. The model waveform is, for example, an arithmetic expression or a table indicating a time-series change in the fall characteristics of the optical signal detector 101. As a result, a pile-up error H' is calculated, and H1 obtained by subtracting the error is output as the detection result and is output as the pixel value to the image display unit 113.

In section 3, a peak position T2 is detected, and H2, which is the peak value of the peak position T2, is output to the photon-counting-type signal processing unit 106. At this time, a process in the subsequent stage is the same as that in section 1.

In section 4, the signal state determination unit 109 calculates T2+T' using the determination time T'. In addition, it is checked from the signal of the integration-type signal processing unit 107 that a signal value is continuous between T2 and T'. At this time, since the continuation of the signal with the determination width d is detected in section 4, the output result of the integration-type signal processing unit 107 is output via the switching unit 112. As a result, for example, it is possible to increase an S/N ratio and to improve the quality of the detection image.

Further, the classification method is not necessarily limited to the method shown in FIG. 4, and may be any classification method using at least the time information of the signal peak. As described above, since the electrical signals including peaks are classified based on at least the time information of the signal peaks, it is possible to appropriately classify the electrical signals.

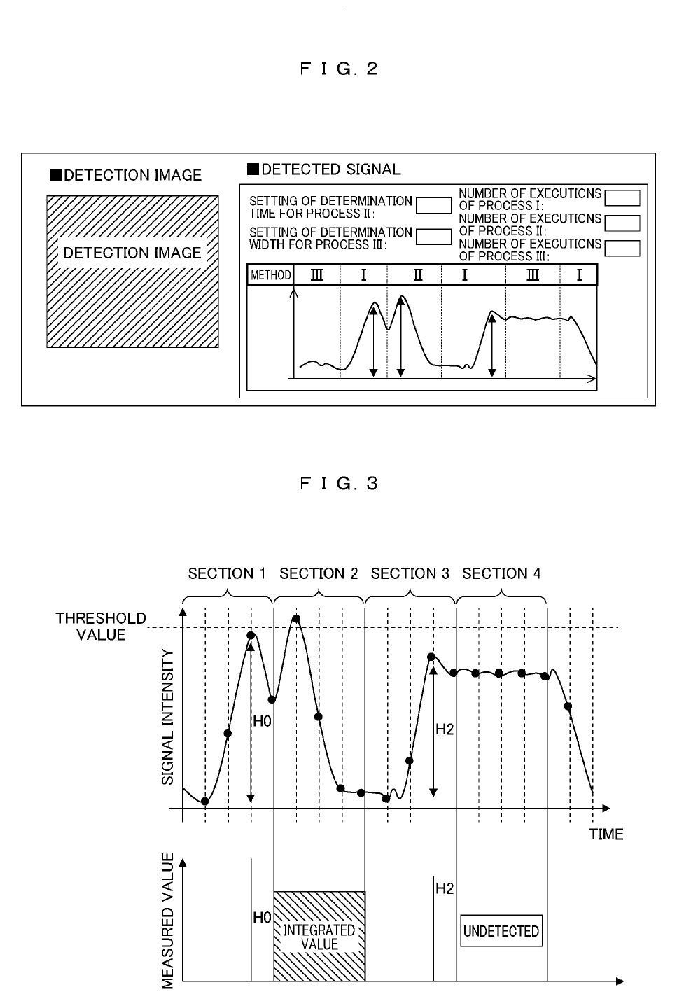

FIG. 2 is a diagram showing an example of the display content of the image display unit 113.

The determination time T' and the determination width d which have been set by the user through the GUI, the number of executions of each process, the detection image, and the signal waveform are displayed on the image display unit 113.

At this time, for example, when the user changes the determination time setting or the determination width setting, the number of executions of each process changes, and the quality (brightness, contrast, or the like) of the detection image also changes with the change in the number of executions of each process. For example, the user can change the determination time setting such that the image quality is improved while visually comparing a plurality of detection images.

FIG. 5 is a flowchart showing a signal processing method according to Example 1.

First, the electrical signal from the optical signal detector 101 is acquired as a target signal (Step S101). Then, a peak position (peak value) of the target signal is detected (Step S102).

Then, the peak time information of the detected peak position is acquired (Step S103). Then, a time obtained by adding the determination time T' to the detected peak time information is calculated and held (Step S104).

Finally, the results of the signal processing are switched in response to the detection of a new peak within the time or the continuation of the target signal (Step S105).

According to Example 1, it is possible to improve the detection rate of the signal processing device using the optical signal detector. Specifically, it is possible to switch between the photon-counting-type signal processing method and the integration-type signal processing method based on the peak time information, to prevent information loss, and to improve the detection rate of the detection result.

### Example 2

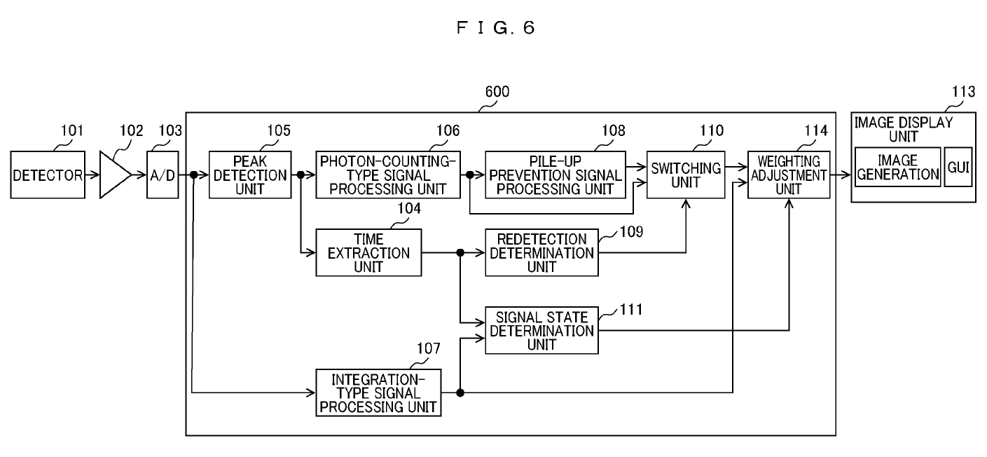

FIG. 6 is a schematic diagram showing an example of a configuration of a signal processing device 600 according to Example 2.

The signal processing device 600 according to Example 2 shown in FIG. 6 differs from the signal processing device 500 according to Example 1 shown in FIG. 1 in that a weighting adjustment unit 114 replaces the switching unit 112 shown in FIG. 1. Since the other configurations are the same as those in FIG. 1, a description thereof will be omitted.

As shown in FIG. 4, in Example 1, the measured values (pixels and detection time) in each section are the photon counting values or the integrated values. In Example 2, the weighting adjustment unit 114 is provided, and the result of combining the photon counting value and the integrated value is used as the measured value.

For example, the states of input signals are classified into state 1 (a discrete pulse state and section 1 in FIG. 4), state 2 (a pile-up state and section 2 in FIG. 4), and state 3 (a saturation state and section 4 in FIG. 4).

In state 1, a coefficient of the photon counting method is 1, and a coefficient of the integration method is 0. In state 2, the coefficient of the photon counting method is 0.5, and the coefficient of the integration method is 0.5. In state 3, the coefficient of the photon counting method is 0, and the coefficient of the integration method is 1.

As described above, the coefficient is set according to the state, and the output results of the photon counting method and the integration method are combined into a pixel value. However, the user can set each coefficient to any value.

According to Example 2, the same effects as various effects described in Example 1 can be obtained. In addition, the user is allowed to change the ratio of the signaling processing methods to any value, which makes it possible to check the influence of the change in the signal processing method on the output results (images and signals).

In the above-described examples, in some cases, processes performed by executing a program are described. Here, a computer executes a program using a processor (for example, a CPU or a GPU) and performs the process defined by the program while using a storage resource (for example, a memory), an interface device (for example, a communication port), and the like. Therefore, the entity that executes the program to perform the process may be the processor.

Similarly, the entity that executes the program to perform the process may be a controller, a device, a system, a computer, or a node that has the processor. The entity that executes the program to perform the process may be a calculation unit and may include a dedicated circuit that performs a specific process. Here, examples of the dedicated circuit include a field programmable gate array (FPGA), an application specific integrated circuit (ASIC), and a complex programmable logic device (CPLD).

The program may be installed on the computer from a program source. The program source may be, for example, a program distribution server or a computer-readable storage medium. When the program source is the program distribution server, the program distribution server may include a processor and a storage resource that stores the program to be distributed, and the processor of the program distribution server may distribute the program to be distributed to other computers. Furthermore, in the examples, two or more programs may be implemented as one program, or one program may be implemented as two or more programs.

According to the above-described examples, it is possible to provide a signal processing device capable of improving detection accuracy.

101: optical signal detector
102: preamplifier circuit
103: A/D converter
104: time extraction unit
105: peak detection unit
106: photon-counting-type signal processing unit
107: integration-type signal processing unit
108: pile-up prevention signal processing unit
109: redetection determination unit
110: switching unit
111: signal state determination unit
112: switching unit
113: image display unit
114: weighting adjustment unit
500: signal processing device
600: signal processing device

What is claimed is:

1. A signal processing device for processing an electrical signal output from an optical signal detector using a processor, the signal processing device comprising:
a peak detection unit;
a photon-counting-type signal processing unit;
an integration-type signal processing unit;
a time extraction unit;
a signal state determination unit; and
an output unit,
wherein the peak detection unit detects a peak value of the electrical signal,
the photon-counting-type signal processing unit detects the peak value included in the electrical signal in a predetermined section to detect the number of photons in the predetermined section and outputs a photon-counting-type signal,
the integration-type signal processing unit integrates a signal included in the electrical signal in the predetermined section to measure a signal quantity and outputs an integration-type signal,
the time extraction unit holds time information of the peak value detected by the peak detection unit,
the signal state determination unit determines whether or not the electrical signal is continuous within an added time obtained by adding a predetermined determination time to the time information of the peak value, and
the output unit outputs at least one of the photon-counting-type signal and the integration-type signal, based on a state signal from the signal state determination unit.

2. The signal processing device according to claim 1,
wherein the output unit constitutes a first switching unit that switches between the photon-counting-type signal and the integration-type signal, based on the state signal from the signal state determination unit, and outputs one of the photon-counting-type signal and the integration-type signal.

3. The signal processing device according to claim 2, further comprising:
a redetection determination unit;
a pile-up prevention signal processing unit; and
a second switching unit;
wherein the redetection determination unit determines whether or not a new pulse is generated within the added time,
the pile-up prevention signal processing unit outputs a pile-up prevention signal obtained by subtracting a pile-up error from the photon-counting-type signal,
the second switching unit switches between the photon-counting-type signal and the pile-up prevention signal, based on a determination signal from the redetection determination unit, and outputs one of the photon-counting-type signal or the pile-up prevention signal, and
the first switching unit switches between the integration-type signal and one of the photon-counting-type signal and the pile-up prevention signal from the second switching unit, based on the state signal from the signal state determination unit, and outputs one of the integration-type signal and the photon-counting-type signal or the pile-up prevention signal.

4. The signal processing device according to claim 1,
wherein the signal state determination unit determines whether or not the electrical signal is continuous within the added time using a predetermined determination width.

5. The signal processing device according to claim 4,
wherein the signal state determination unit uses, as the determination width, a value taking into account noise in the optical signal detector.

6. The signal processing device according to claim 1,
wherein the output unit constitutes a weighting adjustment unit, and
the weighting adjustment unit combines the photon-counting-type signal and the integration-type signal, based on the state signal from the signal state determination unit, and outputs a combined signal.

7. The signal processing device according to claim 6,
wherein the weighting adjustment unit classifies a state of the state signal from the signal state determination unit into a discrete pulse state and a saturated state, and adjusts a weighting coefficient when combining the photon-counting-type signal and the integration-type signal, according to the discrete pulse state and the saturated state.

8. A signal processing system comprising:
the signal processing device according to claim 1;
the optical signal detector;
a preamplifier circuit;
an A/D converter; and
an image display unit,
wherein the optical signal detector converts photons into the electrical signal and transmits the electrical signal to the preamplifier circuit,
the preamplifier circuit amplifies the electrical signal from the optical signal detector,
the A/D converter converts the electrical signal from the preamplifier circuit into a digital signal,
the signal processing device processes the digital signal from the A/D converter, using the processor, to generate a detection image, and
the image display unit displays the detection image generated by the signal processing device.

9. The signal processing system according to claim 8,
wherein the image display unit has a setting screen for displaying the determination time such that a user can set the determination time through the setting screen.

10. The signal processing system according to claim 9,
wherein the image generation unit integrates or averages amplitudes of the peak values included in the predetermined section corresponding to one pixel to calculate a pixel value of the pixel.

11. A signal processing method for processing an electrical signal output from an optical signal detector using a processor, the signal processing method comprising:
a peak detection step;
a photon-counting-type signal processing step;
an integration-type signal processing step;
a time extraction step;
a signal state determination step; and
an output step,
wherein, in the peak detection step, a peak value of the electrical signal is detected,
in the photon-counting-type signal processing step, the peak value included in the electrical signal in a predetermined section is detected to detect the number of photons in the predetermined section, and a photon-counting-type signal is output,
in the integration-type signal processing step, a signal included in the electrical signal is integrated in the predetermined section to measure a signal quantity, and an integration-type signal is output,
in the time extraction step, time information of the peak value detected in the peak detection step is held,
in the signal state determination step, it is determined whether or not the electrical signal is continuous within an added time obtained by adding a predetermined determination time to the time information of the peak value, and
in the output step, at least one of the photon-counting-type signal and the integration-type signal is output based on a state signal obtained in the signal state determination step.

12. The signal processing method according to claim 11,
wherein, in the output step, the photon-counting-type signal and the integration-type signal are switched based on the state signal obtained in the signal state determination step, and one of the photon-counting-type signal and the integration-type signal is output.

13. The signal processing method according to claim 11,
wherein, in the output step, the photon-counting-type signal and the integration-type signal are combined based on the state signal obtained in the signal state determination step, and a combined signal is output.
